(19) United States
(12) Patent Application Publication
(10) Pub. No.: US 2019/0086403 A1
(43) Pub. Date: Mar. 21, 2019

(54) CHROMATOGRAPHIC IMMUNE ASSAY FOR THE DETECTION OF ALLERGIC SENSITIVITIES

(71) Applicant: GLOBALLERGY, LLC, AUSTIN, TX (US)

(72) Inventor: HENRY JOSEPH LEGERE, III, AUSTIN, TX (US)

(21) Appl. No.: 16/192,484

(22) Filed: Nov. 15, 2018

Related U.S. Application Data
(63) Continuation of application No. 15/276,028, filed on Sep. 26, 2016.
(60) Provisional application No. 62/232,264, filed on Sep. 24, 2015.

(51) Int. Cl. G01N 33/543; G01N 33/68; G01N 33/558

(52) U.S. Cl. CPC G01N 33/54386; G01N 33/558; G01N 33/6854

(57) ABSTRACT

An apparatus is provided comprising a conjugate pad, the conjugate pad contains IgG antibodies capable of detecting human IgE antibodies and a nitrocellulose membrane strip including a test zone (T) and a control zone (C), wherein the test zone (T) is coated with a relevant purified antigen cognate to a specific IgE of interest, wherein the coated test zone of the nitrocellulose membrane strip is configured to provide a visual indication of the presence of an anti-antigen IgE antibody in a sample above a predetermined threshold.

### CROSS-REFERENCE TO RELATED APPLICATIONS

This application is a Continuation of U.S. application Ser. No. 15/276,028, filed Sep. 26, 2016, entitled CHROMATOGRAPHIC IMMUNE ASSAY FOR THE DETECTION OF ALLERGIC SENSITIVITIES, which published on Mar. 30, 2017 as U.S. Patent Publication No. US 2017-0089893 A1 (Atty. Dkt. No. RIDL-33300). U.S. application Ser. No. 15/276,028 claims the benefit of U.S. Provisional Application No. 62/232,264, filed Sep. 24, 2015, and entitled CHROMATOGRAPHIC IMMUNE ASSAY FOR THE DETECTION OF ALLERGIC SENSITIVITIES. U.S. application Ser. Nos. 15/276,028 and 62/232,264 and U.S Publication No. US 2017-0089893 A1 are incorporated herein by reference in their entirety.

### TECHNICAL FIELD

The following disclosure relates to a rapid test that is a qualitative lateral flow chromatographic immune assay for the detection of allergies in people, such as food allergies, drug allergies, and environmental allergies.

### SUMMARY

An apparatus is provided comprising a conjugate pad, the conjugate pad contains IgG antibodies capable of detecting human IgE antibodies and a nitrocellulose membrane strip including a test zone (T) and a control zone (C), wherein the test zone (T) is coated with a relevant purified antigen cognate to a specific IgE of interest, wherein the coated test zone of the nitrocellulose membrane strip is configured to provide a visual indication of the presence of an anti-antigen IgE antibody in a sample above a predetermined threshold.

### BRIEF DESCRIPTION OF THE DRAWINGS

For a more complete understanding, reference is now made to the following description taken in conjunction with the accompanying Drawings in which:

FIG. 1 illustrates a diagrammatic representation of one embodiment of an immunoassay test strip; and

FIG. 2 illustrates a diagrammatic representation of one embodiment of an immunoassay test wherein an analyte is tested across multiple test strips.

### DETAILED DESCRIPTION

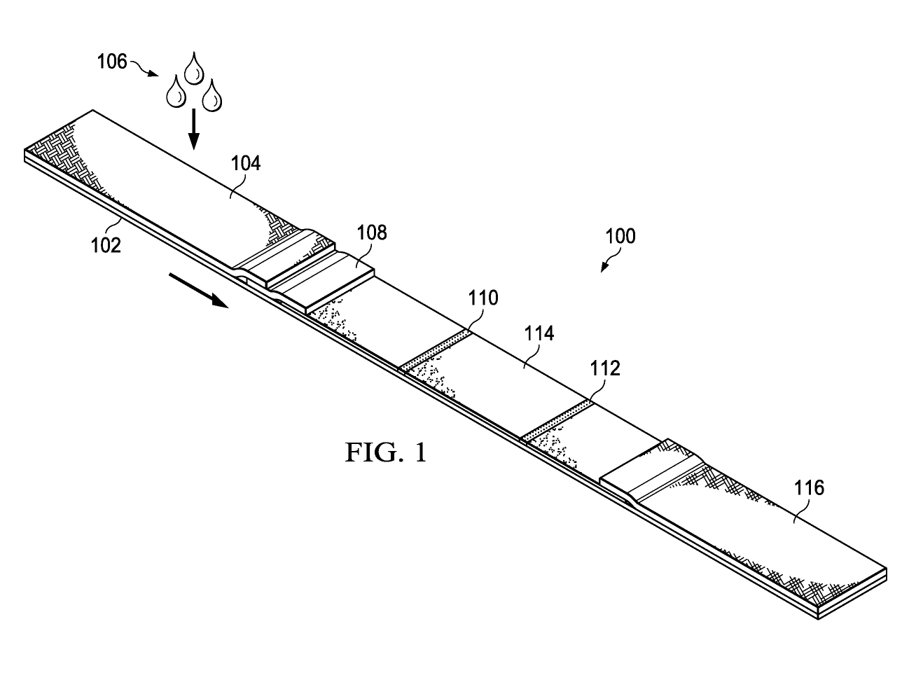

Referring now to FIG. 1, there is illustrated a diagrammatic representation of one embodiment of an immunoassay test strip 100. The immunoassay test strip includes a backing 102, a sample pad 104 to receive a sample 106, a conjugate pad 108, a test line 110 and a control line 112 on a nitrocellulose membrane strip 114, and a wick 116.

The test cassette includes:
1) a colored conjugate pad 108 containing anti-human IgE conjugated with colloid gold or colored latex beads,
2) a nitrocellulose membrane strip 114 containing test zone (T) and a control zone (C). The test zone (T) is precoated with the relevant allergenic antigen (i.e. peanut protein) in question for the detection of anti-antigen IgE antibody and can be in a line or shape.

The test specimen may be a few drops of blood obtained from a finger stick (e.g., finger prick device). The test specimen may be either blood that will be mixed with an adequate amount of buffered solution to create sample analyte (A), or a blood sample that is not diluted or otherwise manipulated, and thus the blood is the sample analyte (A). The analyte (A) is placed in the sample well and migrates along the conjugate pad 108 and further across the coated membrane by capillary action.

The anti-antigen IgE (e.g., anti-peanut IgE antibodies) present in the sample analyte will complex with an antibody capable of detecting human IgE present in the conjugate pad 108 thereby creating an immune complex that will migrate to the test zone and get captured onto the purified antigen (e.g., peanut protein) in the T zone thus giving a qualitative colored response (e.g., a line, shape, plus sign, etc.) when positive. If no antigen-anti-antigen-IgE complexes are present in the analyte, no reaction occurs in the T zone and a qualitative response will not occur. The sample migrates further along the strip until it reaches the control zone where excess anti-IgE antibody-colloidal gold or latex conjugates get bound and produces a qualitative control zone reaction indication that the sample has adequately migrated across the testing membrane as intended.

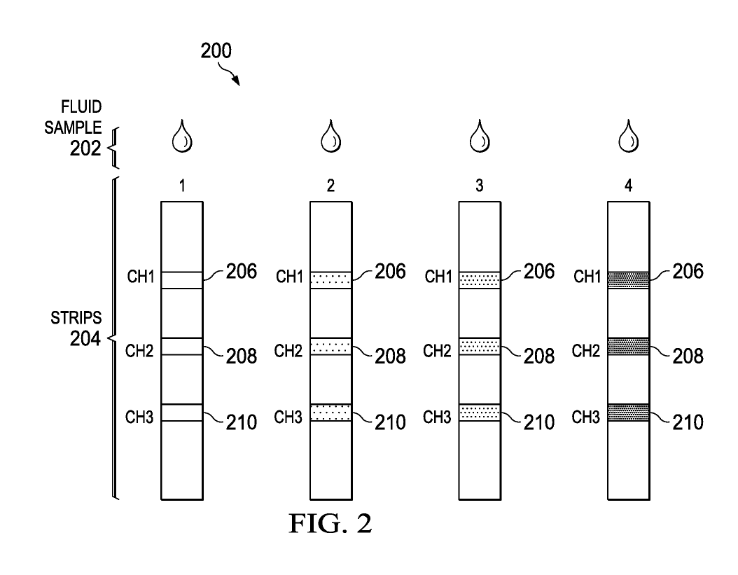

Referring now to FIG. 2, there is illustrated a diagrammatic representation of one embodiment of an immunoassay test 200, wherein a fluid sample 202 is tested across multiple test strips 204. Testing devices can be single allergens or arrays of allergens arranged in panels (CH1 206, CH2 208, CH3 210) of varying combination. For example, configurations for the testing panels can be, but are not limited to: 1) Food 5: Peanut, milk, soy, wheat, egg; 2) Nut and seed panel: almond, cashew, hazelnut, peanut, pecan, walnut, sesame seed, sunflower seed; 3) seafood: crab, lobster, shrimp, salmon, tuna; 4) Pets: cat, dog; 5) Indoor allergens: dust mites, mold mix (alternaria, aspergillus, penicillium, cladosporium), cat, dog; and 6) seasonal allergens: grass (Bermuda, bahia, Johnson, rye, timothy), trees (oak, elm, cedar, mesquite, pine, etc.), weeds (pigweed, ragweed, sage, Russian thistle).

30-40% of adults and children have environmental allergies. Close to 40% of people believe they have food allergies, whereas, only 6-8% actually do. Wait times to get seen by allergists vary from weeks to several months.

Skin tests require coming off of antihistamines for 7-14 days during which people suffer from histamine induced allergic symptoms.

Currently available blood tests to detect allergic sensitization requires a visit to the doctor, a referral to the laboratory, and are frequently not covered by insurance until a significant deductible is met.

Several Lateral Flow Immune Assays (LFIA) have been directed toward identifying proteins, molecules of interest, and even immunoglobulins IgG, IgA, and IgM. IgE is an antibody (immunoglobulin E) that is normally present in the blood freely circulating until it moves into the tissue where it is bound to mast cells through the receptor FcERI (F-C-epsilon-R-one) otherwise known as the high affinity IgE receptor. There is a small amount of IgE bound to IgE receptors (high and low affinity receptors) on basophils, eosinophils, and other cells in the blood and tissues.

Many LFIA systems are geared toward the detection of infectious proteins (e.g. strep, flu, anthrax, etc.). All of the aforementioned tests use a non-human antibody—usually IgG type—e.g., goat IgG antibody directed against a protein of interest to detect the protein of interest from the sample (blood, urine, saliva, sweat, etc.). This antibody complexes with protein of interest and forms a complex that travels across the membrane until it reaches the test zone. In the test zone there is an IgG type antibody directed against IgG from that species of animal. As further described herein, the present detecting apparatus and method use human (patient/consumer-derived) antibodies from the sample and the test zone that contains a humanized antibody directed against the protein of interest that is preconjugated to a detecting substance that results in a visual change.

Summary of Target Antigen:
The target antigens may be proteins, glycoproteins, lipoproteins or other molecular substances capable of eliciting an immune reaction and/or being bound by human specific IgE (sIgE).

LFIA to detect specific IgE:
In the detecting apparatus and method of using the same, the antigens are food proteins or environmental allergenic proteins conjugated to a noble metal, for example, gold, or latex conjugated to antigen (peanut protein) in the test zone, for the purpose of detecting the presence of specific IgE (e.g., anti-peanut IgE in a blood sample from a finger prick). For example, an IgG class antibody (IgG1, IgG2, IgG3, or IgG4) or fragments of those classes of antibodies (fab fragments) whose origin may be any animal species (goat, rat, human, etc.) capable of detecting human IgE (anti-IgE IgG)—a suitable commercially available humanized antibody, such as omaluzimab may be used—may be used to form immune complexes of IgG-anti-IgE-slgE that will migrate to the test zone having selected specific IgE that can bind to the conjugated antigen.

LFIA to detect total IgE (not concerned about specific IgE):
Another embodiment includes using an IgG class antibody (IgG1, IgG2, IgG3, or IgG4) or fragments of those classes of antibodies (fab fragments) whose origin may be any animal species (goat, rat, human, etc.) capable of detecting human IgE (anti-IgE IgG)—a suitable commercially available humanized antibody, such as omaluzimab may be used—that is preconjugated to a detecting molecule that results in a color change when bound to IgE as the target antigen in the test zone.

The apparatus for detection may be a slide supporting the testing service, a cassette based diagnostic test, or a dipstick, and combinations thereof. The apparatus carries a material capable of conjugating to an antigen thereby providing a surface capable of binding an anti-antigen representative of the allergen.

The test results may be in the form of a visual qualitative reading test, a visual semiquantitative format, a reader quantitative assay format, and/or combinations. The time from sample collection to read-out results takes several minutes.

Detection sensitivity: detect specific IgE above 0.35 kUA/L (kilounits of allergen/liter); total IgE detected above 30 IU/mL (international units/milliliter).

The apparatus and method of detecting a sensitivity may be a “one-step” approach from sample to reading without sample dilution or other sample manipulation. The sample may be diluted or endure other sample manipulation, for example the blood sample is diluted with a buffer.

The sample is in the form of blood, saliva or processed tissue sample.

An example of a method of diagnostic testing for allergic sensitivities:
Receive sample, e.g. prick finger
Apply sample to test apparatus and optionally dilute the sample with a suitable buffer
   Optionally dilute the sample with a suitable buffer
Monitor the test apparatus for a change in the test zone, where a visual change in the test zone is a qualitative response indicating the presence of the anti-antigen IgE antibodies present in the sample
Determine the test is concluded when a change has occurred in the control zone, where a visual change in the control zone indicates the sample has adequately migrated across the testing apparatus as intended.

For example, the diagnostic test can be produced in a various formats for different users, such as, but not limited to, consumer/in-home use where the test is purchased through retail channels which will allow individuals to get an immediate, cost-effective test result that can lead to specific avoidance and treatment through follow-up with a medical professional.

The diagnostic test can be provided to and used by hospitals and clinics to provide rapid, on-site test results that are required to prescribe certain medications, such as omaluzimab, by their FDA labels.

This diagnostic assay can be modified to detect the presence of specific IgE in pets.

The present described apparatus, method, and end uses are not limited to the above examples and descriptions. Other embodiments will be apparent to one skilled in the art. As such, the foregoing description merely enables and describes the general uses of the described apparatus and method of using the same. While certain embodiments of the apparatus and method have been described for the purpose of this disclosure, those skilled in the art can make changes without departing from the spirit and scope thereof. Thus, the appended claims define what is claimed.

What is claimed is:

1. A method comprising:
obtaining a sample;
applying the sample to a detecting apparatus, the detecting apparatus including a conjugate pad, the conjugate pad contains IgG antibodies capable of detecting human IgE antibodies, and a nitrocellulose membrane strip including a test zone (T) and a control zone (C), wherein the test zone (T) is coated with a relevant purified antigen of a specific IgE of interest, wherein the coated test zone of the nitrocellulose membrane strip is configured to provide a visual indication of the presence of an anti-antigen IgE antibody in a sample above a predetermined threshold; and
monitoring the detecting apparatus for a visual change in the test zone.

2. The method of claim 1, further comprising determining the method is concluded when a visual change has occurred in the control zone.
